(19) United States
(12) Patent Application Publication
(10) Pub. No.: US 2025/0275996 A1
(43) Pub. Date: Sep. 4, 2025

(54) COMBINATION OF TUNICAMYCIN-TYPE ANTIBIOTIC WITH POLYMYXINS AND USES THEREOF

(71) Applicant: The United States of America, as Represented by the Secretary of Agriculture, Washington, DC (US)

(72) Inventors: NEIL P. PRICE, ELDELSTEIN, IL (US); MICHAEL A. JACKSON, MORTON, IL (US)

(21) Appl. No.: 18/591,513

(22) Filed: Feb. 29, 2024

(51) Int. Cl. A61K 31/7072; A61K 31/395; A61K 31/546; A61P 31/04

(52) U.S. Cl. CPC A61K 31/7072; A61K 31/546; A61K 38/12; A61L 2/16; A61P 31/04; A61L 2101/44

(57) ABSTRACT

Described are antibiotic compositions effective against Gram-negative bacteria, the compositions comprising a polymyxin in combination with a tunicamycin or a modified tunicamycin, optionally in combination with a β-lactam antibiotic. Use of these compositions to kill or inhibit Gram-negative bacteria, treat Gram-negative bacterial infections, or disinfect objects or surfaces are described.

### FIELD

This disclosure relates to the biomedical field, and in particular relates to compositions comprising a polymyxin and a tunicamycin-type antibiotic, and methods to treat infections using such compositions.

### BACKGROUND

Gram-negative bacteria (GNB) are among the world's most significant public health problems due to their high resistance to antibiotics. Two large groups, Enterobacteriaceae and the non-fermenters, are responsible for most clinical isolates; nevertheless, other clinically concerning gram-negative organisms exist, including but not limited to Neisseria, Haemophilus spp., Helicobacter pylori, and Chlamydia trachomatis. The main difference between GNB and Gram-positive bacteria (GPB) is that the GNB have two cellular membranes. The extra, outermost membrane of GNB is quite unique, and composed of lipopolysaccharide. The inner membrane of GNB is fundamentally similar to the one around GPB.

Polymyxins comprise a class of antibiotics targeting gram-negative bacterial infections. Polymyxin B and Polymyxin E (also known as colistin) are the two drugs within this antibiotic class used primarily in clinical practice. They are FDA approved for serious infections with multidrug-resistant gram-negative bacteria, especially those caused by Enterobacteriaceae, Pseudomonas aeruginosa, and Acinetobacter baumannii. Polymyxins are often the only effective antibiotic agent against multidrug-resistant organisms, particularly carbapenem-resistant Enterobacteriaceae.

Polymyxin B and E are well-established antibiotics used in topical creams, which work on GNB by clumping together the lipopolysaccharide in their outer membrane. This makes the outer membrane leaky. Discovered in the 1940s, Polymyxin B consists of basic polypeptides of about eight amino acids that have cationic detergent action on cell membranes. All gram-positive bacteria, fungi, and the gram-negative cocci, are resistant. Given the narrow therapeutic indices of polymyxins, a strategy for overcoming attenuated polymyxin susceptibility without increasing polymyxin exposure is the use of polymyxins in combination with other agents. Polymyxins have a unique mechanism of action that involves disruption of the outer membrane integrity of gram-negative bacteria, which may enhance the activity of other antibiotic classes. Despite promising in vitro results, the usefulness of synergistic polymyxin combinations in the clinical setting remains controversial.

Because bacteria are becoming resistant to antibiotics currently on the market, a need exists for compositions that are effective in controlling bacteria.

### SUMMARY

The disclosure relates to antibacterial compositions comprising at least one polymyxin and tunicamycin or a modified tunicamycin, and optionally comprising a β-lactam antibiotic. The disclosure also relates to methods of killing or inhibiting bacteria, or of treating a subject of infections caused by bacteria using such compositions.

In an embodiment, the disclosure relates to an antibacterial composition comprising at least one polymyxin and at least one tunicamycin or a salt thereof, or comprising at least one polymyxin and at least one modified tunicamycin or a salt thereof. In some embodiments of the disclosure, the antibacterial composition further comprises a β-lactam antibiotic.

In some embodiments of the disclosure, the antibacterial composition comprises a polymyxin and a modified tunicamycin or salt thereof; where the modified tunicamycin or salt thereof comprises Formula I

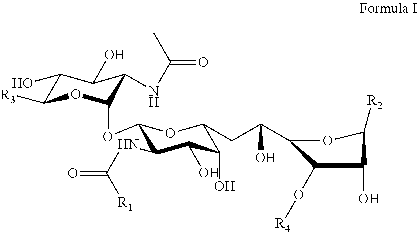

where R₂ is X or Y

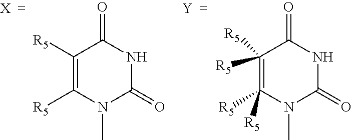

where, R₁ is independently (i) CH₃-(CH₂)n-CH₂-CH₂- and n is independently any integer from 9 to 15, or (ii) CH₃-CH₂-CH(CH₃)(CH₂)n-CH₂-CH₂- and n is independently any integer from 6 to 12; where R₃ is independently HOCH₂-, an alkyl hydrocarbon, or a non-alkyl C₁₋₁₀ hydrocarbon; where R₄ is independently H or P-(O)(OH)₂; and where R₅ is independently H, F, Cl, Br, I, an alkyl hydrocarbon, or a non-alkyl C₁₋₁₀ hydrocarbon; or where the modified tunicamycin or salt thereof comprises Formula II

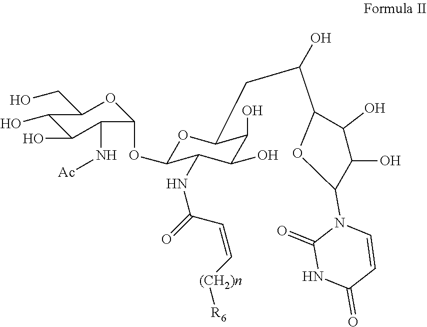

where n is independently any integer from 7 to 13; and R₆ is a C1 or C2 straight chain, an iso-branched chain, an anteiso-branched chain, a tert-butyl-branched chain, a cyclopropane, a cyclobutene, a cyclopentane, a cyclohexane, a cycloheptane, an unsaturated cyclohexene, or an unsaturated cyclopentene.

In some embodiments of the disclosure, the polymyxin in the Gram-negative bacteria-killing composition is polymyxin B or polymyxin E. In some embodiments of the disclosure, the β-lactam antibiotic in the Gram-negative bacteria-killing composition is a penicillin, a cephalosporin, a monobactam, a carbapenem, or a combination thereof. In some embodiments of the disclosure, the Gram-negative killing composition comprises a carrier.

In an embodiment, the disclosure relates to a method of killing Gram-negative bacteria in or on a subject by administering an effective dose of a composition as taught herein to a subject in need thereof. A further embodiment of the disclosure is a method of inhibiting or treating a bacterial infection caused by Gram-negative bacteria in a subject in need thereof, the method comprising administering to the subject a therapeutically effective amount of a composition comprising a polymyxin in combination with a tunicamycin, a modified tunicamycin, or a salt thereof. In an embodiment, the disclosure relates to a method of disinfecting an object or a surface by applying a composition as taught herein alone or in combination with a carrier and/or other components to inhibit growth or kill Gram-negative bacteria present on the object or surface. In some embodiments of the disclosure, the subject is an animal, a plant, or an insect.

### BRIEF DESCRIPTION OF THE DRAWINGS

FIG. 1A to FIG. 1C depict the chemical structure of modified tunicamycins. FIG. 1A shows the structure of single reduced tunicamycins (referred to as Tun-R1); FIG. 1B shows the structure of double reduced tunicamycins (referred to as Tun-R2); and FIG. 1C shows the structure of an omega tunicamycin (a tunicamycin comprising an integrated small carboxylic acid at the omega position).

FIG. 2A and FIG. 2B depict polymyxin chemical structures. The structure of polymyxin B is shown in FIG. 2A, where R is H or CH₃. The structure of polymyxin E is shown in FIG. 2B.

FIG. 3A and 3B depict images taken after 24 hour incubation at 30° C. of plates stained with resazurin and inoculated with bacteria or yeast and treated with polymyxin B in a dilution series, or DMSO alone. The wells in the plate shown in FIG. 3A were inoculated with E. coli (six left columns) or Pseudomonas aeruginosa (six right columns). The wells in the plate shown in FIG. 3B were inoculated with Bacillus (six left columns) or yeast (six right columns). Indicated at the top of the plates are the treatments (DMSO alone (control) or Polymyxin B). The microorganism type is indicated at the bottom of the plates. The antibiotic dilution in μg/mL is indicated to the right of the plates. The minimum inhibitory concentrations (MIC₉₀) are indicated by white horizontal bars.

FIG. 4A to FIG. 4D depict images taken after 24 hour incubation at 37° C. of plates stained with resazurin and inoculated with bacteria and treated with Tun or TunR2 in the presence or absence of polymyxin B in a dilution series, or DMSO alone. The wells in the plates shown in FIG. 4A and FIG. 4B were inoculated with E. coli. The wells in the plates shown in FIG. 4C and FIG. 4D were inoculated with P. aeruginosa. The wells in the six right columns were treated without polymyxin, and the wells in the six left columns were treated with polymyxin. Wells in columns 1, 2, 7, and 8 were treated with DMSO; wells in columns 3, 4, 9, and 10 were treated with Tun; wells in columns 5, 6, 11, and 12 were treated with TunR2. The antibiotic dilution in μg/mL is indicated to the right of the plates.

FIG. 5A to FIG. 5D depict images taken after 48 hour incubation at 37° C. of plates stained with resazurin and inoculated with bacteria and treated with Tun or TunR2 in the presence or absence of polymyxin B in a dilution series, or DMSO alone. The wells in the plates shown in FIG. 5A and FIG. 5B were inoculated with E. coli. The wells in the plates shown in FIG. 5C and FIG. 5D were inoculated with P. aeruginosa. The wells in the six right columns were treated without polymyxin, and the wells in the six left columns were treated with polymyxin. Wells in columns 1, 2, 7, and 8 were treated with DMSO; wells in columns 3, 4, 9, and 10 were treated with Tun; wells in columns 5, 6, 11, and 12 were treated with TunR2. The antibiotic dilution in μg/mL is indicated to the right of the plates.

FIG. 6A to FIG. 6D depict images taken after 7 day incubation at 37° C. of plates stained with resazurin and inoculated with bacteria and treated with Tun or TunR2 in the presence or absence of polymyxin B in a dilution series, or DMSO alone. The wells in the plates shown in FIG. 6A and FIG. 6B were inoculated with E. coli. The wells in the plates shown in FIG. 6C and FIG. 6D were inoculated with P. aeruginosa. The wells in the six right columns were treated without polymyxin, and the wells in the six left columns were treated with polymyxin. Wells in columns 1, 2, 7, and 8 were treated with DMSO; wells in columns 3, 4, 9, and 10 were treated with Tun; wells in columns 5, 6, 11, and 12 were treated with TunR2. The antibiotic dilution in μg/mL is indicated to the right of the plates.

FIG. 7A and FIG. 7B depict images taken after 24 hours incubation at 37° C. of a plate inoculated with Serratia marcescens or E. coli in TYG and treated with Tun or TunR2 in a dilution series alone or with the addition of Polymyxin B. FIG. 8A shows an image of wells containing S. marcescens, an FIG. 8B shows an image of wells containing E. coli. The wells on columns 1 to 6 were treated in the absence of Polymyxin B, and the wells on columns 7 to 12 were treated in the presence of Polymyxin B. Wells in columns 1, 2, 7, and 8 were treated with DMSO (control), wells in columns 3, 4, 9, and 10 were treated with Tun; wells in columns 5, 6, 11, and 12 were treated with TunR2. The MIC are marked with white lines. The treatments are indicated at the top and bottom of the image, and the Tun or TunT2 dilution in μg/mL is indicated to the right of the plate.

FIG. 8 depicts an image taken after 48 hours incubation at 37° C. of a plate inoculated with P. aeruginosa in TYG and treated with Cefquinome in a dilution series alone or with the addition of Polymyxin B, TurnR2, or both. The wells on the first column on the left were treated with DMSO (control), wells 2 and 3 were treated with Cefquinome; wells 4 to 6 were treated with Cefquinome with 2.5 μg/mL Polymyxin B; wells 7 to 9 were treated with Cefquinome with 6.25 μg/mL TunR2; wells 10 to 12 were treated with Cefquinome with 2.5 μg/mL Polymyxin B and 6.25 μg/mL TunR2. The MIC are marked with white lines, the MIC for Cefquinome (10.0 μg/mL) is indicated by a white arrow on the left of the plate, and the MIC for Cefquinome with Polymyxin B and TunR2 is indicated by a black arrow on the right of the plate. The treatments are indicated at the top of the image, and the Cefquinome dilution in μg/mL is indicated to the right of the plate.

### DETAILED DESCRIPTION

The present disclosure relates to Gram-negative bacteria-killing compositions comprising at least one polymyxin and at least one tunicamycin or modified tunicamycin, and optionally comprising a lactam antibiotic. The disclosure also relates to methods to treat a subject in need thereof of infections caused by gram-negative bacteria using such compositions.

Gram-negative bacteria are naturally resistant to many antibiotics due to the presence of the outer membrane, a unique asymmetric bilayer with phospholipids in the inner leaflet and lipopolysaccharide (LPS) in the outer leaflet. LPS is a large glycolipid containing multiple fatty acyl chains that comprise the hydrophobic region of the outer leaflet. Phosphate groups attached to the core sugars bind divalent metal cations to form a well-ordered polyelectrolyte barrier. Many antibacterial agents are unable to penetrate this barrier, and many of those agents that somehow cross the barrier are immediately exported by multi-drug efflux pumps.

The disclosure shows the surprising discovery that combination of polymyxin with a tunicamycin or a modified tunicamycin has a powerful synergistic bactericidal effect against Gram-negative bacteria. Based on the synergistic effect achieved when the compounds are in combination, an effective dosage of the tunicamycin or modified tunicamycin and/or the polymyxin in the combination is reduced relative to a dosage based on either drug alone. The disclosure shows that when combined with tunicamycin or a modified tunicamycin reduced doses of polymyxin synergistically act to kill polymyxin- and/or tunicamycin- and/or modified tunicamycin-resistant Gram-negative bacteria.

Gram-negative bacteria are a group of bacteria characterized by their cell envelopes, which are composed of a thin peptidoglycan cell wall sandwiched between and inner cytoplasmic cell membrane and a bacterial outer membrane. The outer membrane contains LPS, which consist of lipid A, a core polysaccharide, and O antigen in its outer leaflet and phospholipids in its inner leaflet.

The compositions and methods of described herein may be used to treat any Gram-negative bacteria. Exemplary Gram-negative bacteria are the proteobacteria such as Escherichia coli, Salmonella, Shigella, and other Enterobacteriaceae, Pseudomonas, Moraxella, Helicobacter, Stenotrophomas, Bdellovibrio, and Legionella. Other Gram-negative bacteria include Neisseria gonorrhoeae, N. meningitides, M. catarrhalis, Haemophilus influenzae, Klebsiella pneumoniae, Legionella pneumophila, Pseudomonas aeruginosa, Proteus mirabilis, Enterobacter cloacae, Serratia marcescens, Helicobacter pylori, Salmonella enteritidis, Salmonella typhi, Burkholderia cenocepacia, and Acinetobacter baumannii.

Gram-negative bacteria known to infect plants include Pseudomonas syringae, Ralstonia solanacearum, Xanthomonas oryzae, X. campestris, X. axonopodis, Erwinia amylovora, Xylella fastidiosa, Dickeya dadantii, Pectobacterium carotovorum, Pseudomonas savastanoi, and Candidatus Liberibacter. Gram-negative bacteria known to infect insects include Yersinia entomophaga, Pseudomonas entomophila, Photorhabdus asymbiotica, Enterobacter cloacae, Pectobacterium carotovorum, Micrococcus luteum, and Provencia heimbachae. Serratia marcescens is an environmental bacterium that frequently acts as an opportunistic pathogen of insects.

According to P. J. Bergen (2019, “Rational Combinations of Polymyxins with Other Antibiotics,” in: Li, J., Nation, R., Kaye, K. (eds) Polymyxin Antibiotics: From Laboratory Bench to Bedside, Adv. Exp. Med. Biol. vol 1145. Springer, Cham. https://doi.org/10.1007/978-3-030-16373-0_16), polymyxin combination therapy has been suggested as a means to increase antimicrobial activity and reduce development of polymyxin resistance. F. Perez et al (2019, “Polymyxins: To Combine or Not to Combine,” Antibiotics 8(2), 38; doi:10.3390/antibiotics8020038) assess studies on the clinical efficacy of polymyxin combination therapy against infections caused by carbapenem-resistant Enterobacteriaceae (CRE), carbapenem-resistant Acinetobacter baumannii (CRAB), and carbapenem-resistant P. aeruginosa (CRPA). Perez concludes that strategies must be developed to optimize polymyxin-based treatments, informed by in vitro hollow fiber models, careful clinical observations, and high-quality evidence from appropriately designed trials.

Not intending to be theory-bound, it is thought that low concentrations of polymyxin make the Gram-negative bacterial membrane permeable, so that combinations of polymyxin with tunicamycin or a modified tunicamycin will be effective against Gram-negative bacteria.

The effectiveness of mixtures of tunicamycin or a tunicamycin derivative (TunR2) was evaluated on Pseudomonas aeruginosa and E. coli, two Gram-negative bacteria identified by the World Health Organization as critical pathogens for which new antibiotics are required. TunR2 alone is inactive on both of these species. First, the MIC of polymyxin was determined for these bacteria (0.625 ug/mL for E. coli, and 5.0 ug/ml for Pseudomonas). Then a sublethal amount of polymyxin (MIC/2, 0.313 μg/mL for E. coli, and 2.5 μg/mL for Pseudomonas) was used in all of the microtiter plate wells—plus a dilution series of either TunR2 or natural tunicamycin. After 24 hours growth all of the wells were bacteria-free for the TunR2/polymyxin and tunicamycin/polymyxin combinations, but bacteria grew in all the wells without the polymyxin component. After 48 hours growth the bacterial growth started to come back, and by 7 days there was growth of E. coli in the polymyxin/tunicamycin wells—but the TunR2/polymyxin wells were still completely clear. This combination was also effective against the Pseudomonas aeruginosa (MIC˜10 ug/ml TunR2) which is a notoriously difficult bacterium to kill. Thus, in the instant disclosure the TunR2/polymyxin combinations are shown to be effective as an antibiotic treatment against Gram negative bacteria, whereas the TunR2 alone is not. The TunR2/polymyxin in combination may enhance beta-lactams against GN pathogens. This is very important because according to the WHO beta-lactam-resistant GN pathogens are their major concern to animal and human health. The TunR2/polymyxin combination may be able to overcome this resistance. The findings shown here provide evidence of natural tunicamycin/polymyxin or TunR2/polymyxin antibiotic combinations for effective topical or intravenous administration for Gram negative bacterial infection treatments in veterinary, food safety, and/or medical applications.

The tunicamycins (TUN) are a family of potent and naturally occurring nucleoside antibiotics that have shown efficacy in curtailing the growth of numerous bacterial species. U.S. Pat. No. 10,513,533 discloses tunicamycin-related compounds with antibacterial activity having an acyl chain double bond and/or an uracil ring double bond reduced. Several structural modifications in TUN have been instrumental in improving its antibiotic value while reducing the TUN toxic effects to eukaryotic cells. For example, the modified TUN variants, TunR1 and TunR2, are considerably less toxic to eukaryotes but maintain potent antibacterial activity (see, for example, NPJ Price, et al., 2019, “Synergistic enhancement of beta-lactam antibiotics by modified tunicamycin analogs TunR1 and TunR2,” J. Antibiot. (Tokyo) 72: 807-815; and NP Price et al., 2017, “Modified tunicamycins with reduced eukaryotic toxicity that enhance the antibacterial activity of β-lactams,” J Antibiot. (Tokyo) 70:1070-1077).

Polymyxin compounds are antibiotics such as those that are naturally produced by the P. polymyxa Gram-positive bacteria. Polymyxins may be useful in the treatment of Gram-negative bacterial infections and function by disruption of the bacterial cell membrane. They are often neurotoxic and nephrotoxic, so they are commonly used as a last resort of antibiotics if other treatments are ineffective, such as in the case of multi-drug resistant infections. Polymyxins include polymyxin A, polymyxin B, polymyxin C, polymyxin D, and polymyxin E (also known as colistin), the structures of which are shown below.

This disclosure describes methods of treatment for bacterial infections by administering a pharmaceutical composition. The pharmaceutical composition can be formulated with a pharmaceutically-acceptable carrier or excipient. A pharmaceutically-acceptable carrier or excipient refers to a carrier (e.g., carrier, media, diluent, solvent, vehicle, etc.) which does not significantly interfere with the biological activity or effectiveness of the active ingredient(s) of a pharmaceutical composition, and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types).

A pharmaceutical composition is typically formulated to be compatible with its intended route of administration. The composition may be administered orally, nasally, enterally, parenterally, intramuscularly, intravenously, subcutaneously, intradermally, rectally, vaginally, topically, ocularly, pulmonarily, or by contact application. In some instances, agents can be formulated by combining the active compounds with pharmaceutically-acceptable carriers well known in the art. Such carriers enable the compounds of the invention to be formulated as a powder, tablet, pill, capsule, lozenge, liquid, gel, syrup, slurry, suspension, and the like. It is recognized that some pharmaceutical compositions, if administered orally, must be protected from digestion. This is typically accomplished either by complexing the protein with a composition to render it resistant to acidic and enzymatic hydrolysis or by packaging the protein in an appropriately resistant carrier such as a liposome. Suitable excipients for oral dosage forms include, for example, fillers such as sugars, including lactose, sucrose, mannitol, or sorbitol; cellulose preparations such as, for example, starch, gelatin, gum tragacanth, methyl cellulose, hydroxypropylmethyl cellulose, sodium carboxymethylcellulose, and/or polyvinylpyrrolidone (PVP). Disintegrating agents may be added, for example, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers.

For topical application, a pharmaceutical composition may be formulated in a suitable ointment, lotion, gel, or cream containing the active components suspended or dissolved in one or more pharmaceutically-acceptable carriers suitable for use in such compositions.

The pharmaceutical compositions described herein can be administered to a subject (e.g., an animal, a plant, an insect, or an object) in a variety of ways. The compositions must be suitable for the individual receiving the treatment and the mode of administration. Furthermore, the severity of the infection to be treated affects the dosages and routes. The pharmaceutical compositions taught herein can be administered orally, sublingually, parenterally, intravenously, subcutaneously, intramedullary, intranasally, as a suppository, using a flash formulation, topically, intradermally, subcutaneously, via pulmonary delivery, via intra-arterial injection, ophthalmically, optically, intrathecally, or via a mucosal route. The compositions taught herein may also be applied topically or on a surface.

Compositions disclosed herein can be in a form suitable for topical use such as, for example, an aerosol, cream, ointment, lotion, dusting powder, mouth wash, gargle, and the like. Further, the compositions can be in a form suitable for use in transdermal devices. These formulations can be prepared, utilizing the compositions described herein via conventional processing methods. As an example, a cream or ointment is prepared by mixing hydrophilic material and water, together with about 5 wt % to about 10 wt % of the active agents (combinations taught herein) to produce a cream or ointment having a desired consistency.

As a disinfectant, the antibacterial compositions containing a polymyxin and a tunicamycin or modified tunicamycin can be made into a solution, suspension, emulsion, gel, or spray that can be wiped on, sprayed on, poured on, etc., onto a surface. It also can be a solution, suspension, or gel into which a body part or object is dipped. It can also be made into a solution, suspension, emulsion, gel, or similar type of compound that can be applied to a subject or part of a subject. Such disinfecting solutions, suspensions, emulsions, gels, or sprays containing an antibacterial composition comprising at least one polymyxin and at least one tunicamycin or a salt thereof, or comprising at least one polymyxin and at least one modified tunicamycin or a salt thereof can be applied to medical or veterinarian instruments, food-preparing surfaces.

In general, the dosage of a pharmaceutical composition or the active agent in a pharmaceutical composition may be in the range of from about 1 pg to about 1 kg (e.g., 1 pg-10 pg, e.g., 2 pg, 3 pg, 4 pg, 5 pg, 6 pg, 7 pg, 8 pg, 9 pg, 10 pg, e.g., 10 pg-100 pg, e.g., 20 pg, 30 pg, 40 pg, 50 pg, 60 pg, 70 pg, 80 pg, 90 pg, 100 pg, e.g., 100 pg-1 ng, e.g., 200 pg, 300 pg, 400 pg, 500 pg, 600 pg, 700 pg, 800 pg, 900 pg, 1 ng, e.g., 1 ng-10 ng, e.g., 2 ng, 3 ng, 4 ng, 5 ng, 6 ng, 7 ng, 8 ng, 9 ng, 10 ng, e.g., 10 ng-100 ng, e.g., 20 ng, 30 ng, 40 ng, 50 ng, 60 ng, 70 ng, 80 ng, 90 ng, 100 ng, e.g., 100 ng-1 μg, e.g., 200 ng, 300 ng, 400 ng, 500 ng, 600 ng, 700 ng, 800 ng, 900 ng, 1 μg, e.g., 1-10 μg, e.g., 1 μg, 2 μg, 3 μg, 4 μg, 5 μg, 6 μg, 7 μg, 8 μg, 9 μg, 10 μg, e.g., 10 μg-100 μg, e.g., 20 μg, 30 μg, 40 μg, 50 μg, 60 μg, 70 μg, 80 μg, 90 μg, 100 μg, e.g., 100 μg-1 mg, e.g., 200 μg, 300 μg, 400 μg, 500 μg, 600 μg, 700 μg, 800 μg, 900 μg, 1 mg, e.g., 1 mg-10 mg, e.g., 2 mg, 3 mg, 4 mg, 5 mg, 6 mg, 7 mg, 8 mg, 9 mg, 10 mg, e.g., 10 mg-100 mg, e.g., 20 mg, 30 mg, 40 mg, 50 mg, 60 mg, 70 mg, 80 mg, 90 mg, 100 mg, e.g., 100 mg-1 g, e.g., 200 mg, 300 mg, 400 mg, 500 mg, 600 mg, 700 mg, 800 mg, 900 mg, 1 g, e.g., 1 g-10 g, e.g., 2 g, 3 g, 4 g, 5 g, 6 g, 7 g, 8 g, 9 g, 10 g, e.g., 10 g-100 g, e.g., 20 g, 30 g, 40 g, 50 g, 60 g, 70 g, 80 g, 90 g, 100 g, e.g., 100 g-1 kg, e.g., 200 g, 300 g, 400 g, 500 g, 600 g, 700 g, 800 g, 900 g, 1 kg).

The dose may also be administered as in a unit dose form or as a dose per mass or weight of the patient from about 0.01 mg/kg to about 1000 mg/kg (e.g., 0.01-0.1 mg/kg, e.g., 0.02 0.03mg/kg, 0.04 mg/kg, 0.05 mg/kg, 0.06 mg/kg, 0.07 mg/kg, 0.08 mg/kg, 0.09 mg/kg, 0.1 mg/kg, e.g., 0.1-1 mg/kg, e.g., 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, e.g., 1-10 mg/kg, e.g., 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, e.g., 10-100 mg/kg, e.g., 20 mg/kg, 30 mg/kg, 40 mg/kg, 50 mg/kg, 60 mg/kg, 70 mg/kg, 80 mg/kg, 90 mg/kg, 100 mg/kg, e.g., 100-1000 mg/kg, e.g., 200 mg/kg, 300 mg/kg, 400 mg/kg, 500 mg/kg, 600 mg/kg, 700 mg/kg, 800 mg/kg, 900 mg/kg, 1000 mg/kg). The dose may also be administered as a dose per mass or weight of the patient per unit day (e.g., 0.1-10 mg/kg/day).

The dosage regimen may be determined by the clinical indication being addressed, as well as by various patient variables (e.g., weight, age, sex) and clinical presentation (e.g., extent or severity of disease). Furthermore, it is understood that all dosages may be continuously given or divided into dosages given per a given time frame. The composition can be administered, for example, every hour, day, week, month, or year.

The compositions of this invention may be prepared into a kit. For example, the kit may contain a composition (e.g., comprising an aminocoumarin compound and/or a polymyxin compound) and instructions for administering the composition (e.g., the aminocoumarin compound and/or the polymyxin compound) to treat a bacterial infection.

As the methods of the invention described herein include treating or preventing Gram-negative bacterial infections, it may be useful to design combination therapies comprising two or more pharmaceutical agents (e.g., a tunicamycin, a modified tunicamycin, or a salt thereof, and a polymyxin compound or salt thereof). The two or more agents may be administered sequentially, or at substantially the same time. If administered sequentially, the second agent may be administered about 1 minute, 1 hour, 1 day, or 1 week after the first agent. The two or more agents may be administered in the same formulation or as separate formulations.

The combination therapies of the invention may be useful in providing synergistic effects of the two or more pharmaceutical agents. For example, the minimum inhibitory concentration (MIC) or minimum bactericidal concentration (MBC) of a certain drug may be lowered upon administration with a second agent. Additionally, the cumulative treatment effect of two drugs in combination may be greater than the sum of the treatment effects of each individual drug. This behavior may be beneficial by lowering the amount of drug required to treat certain indications. When a drug exhibits toxicity, it is preferable to use the lowest dosage possible to achieve a treatment effect in order to minimize detrimental side effects while still maintaining efficacy. In some embodiments, the effects of the combination therapy will be synergistic, but the side effects will not.

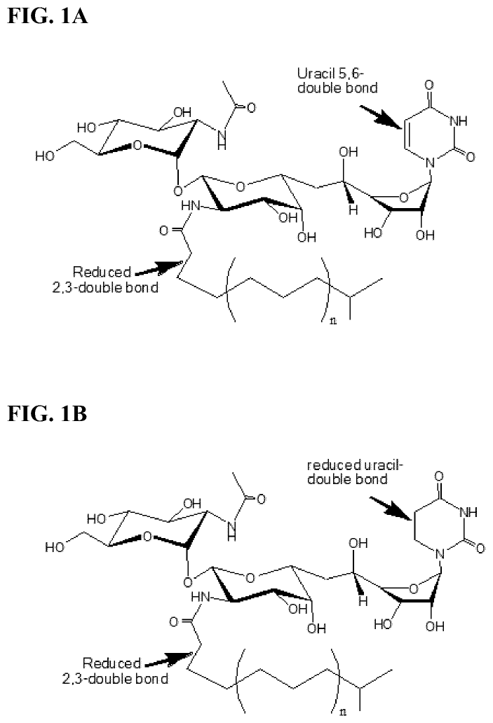

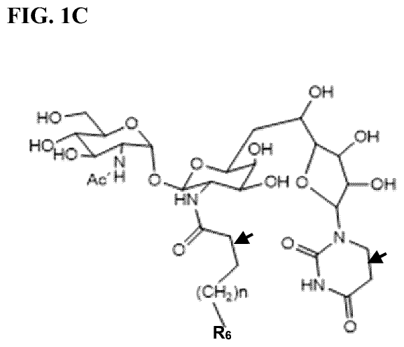

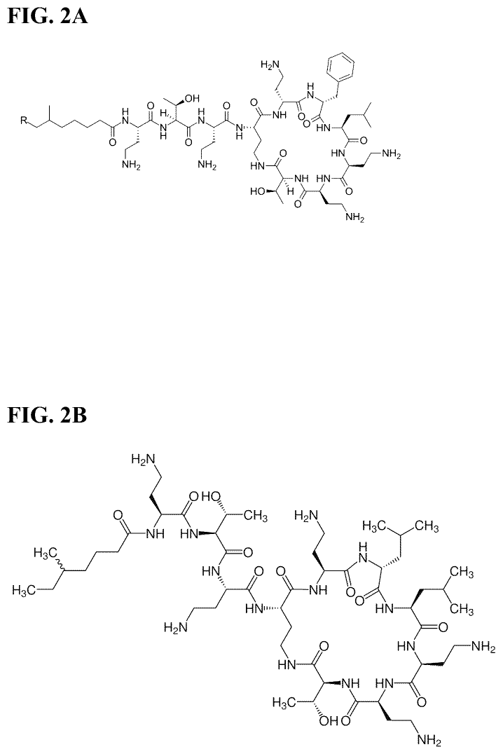

As used herein, the terms “tunicamycin-derived” and “modified tunicamycin” are used interchangeably and refer to a modified tunicamycin or salt thereof including single reduced tunicamycins in which the fatty acid acyl 2″′, 3″′-double bond or the uracil 5,6 double bond are reduced, and have the general structure as shown in FIG. 1A, wherein n is an integer from 1 to 20; double reduced tunicamycins in which the fatty acid acyl 2″′, 3″′ double bond and the uracil 5,6 double bond are reduced, and have the general structure as shown in FIG. 1B, wherein n is an integer from 1 to 20; N-acyl-tunicamycin variants, with the general structure as shown in FIG. 1C, wherein R₆ is a straight chain, an iso-branched chain, an anteiso-branched chain, a tert-butyl-branched chain, a cyclopropane, a cyclobutene, a cyclopentane, a cyclohexane, or a cycloheptane. Compounds and salts thereof include those described in U.S. Pat. No. 10,513,533 and US Patent publication 2023/0320354, and any compounds, prodrugs, or analogs with structural similarity to any of these compounds.

As used herein, the term “alkyl hydrocarbon” refers to a branched saturated hydrocarbon chain. The alky hydrocarbon may contain any number of carbon atoms, for example, may contain 18 carbons, 17 carbons, 16 carbons, 15 carbons, 14 carbons, 13 carbons, 12 carbons, 11 carbons, 10 carbons, 9 carbons, 8 carbons, 7 carbons, or 6 carbons.

As used herein, the term “non-alkyl C₁₋₁₀ hydrocarbon” refers to a non-branched, saturated or unsaturated hydrocarbon of 1 to 10 carbons.

As used herein, the term “polymyxin compound” refers to antibiotic compounds and salts thereof including polymyxin A, polymyxin B, polymyxin C, polymyxin D, and polymyxin E (also known as colistin), and any compounds, prodrugs, or analogs with structural similarity to these compounds. Polymyxin compounds include those described in U.S. Pat. Nos. 10,047,126; 9,763,996; 9,096,649; 9,090,669; 9,067,974; 8,680,234; 8,642,535; 8,329,645; 8,329,430; 8,193,148; 7,890,380; 7,807,637; 7,507,718; 6,579,696; 6,380,356; and 5,177,059; and US Patent Publication Nos. 20170073373; 20160287661; 20120316105; and 20090203881. Polymyxin compounds also include molecules having similar structures and antibacterial properties and that function with a similar mechanism as any of the compounds listed above.

As used herein, the term “bacterial infection” refers to the invasion of an individual's cells, tissues, and/or organs by bacteria (e.g., Brucella; Borrelia (Lyme disease); Campylobacter spp.; Bartonella henselae; Vibrio cholerae; Escherichia coli; Erwinia (apples/pears); Haemophilus influenzae; Klebsiella; Enterobacter; Serratia; Legionella pneumophila; Bordetella pertussis; Yersinia pestis; Pseudomonas spp.; Salmonella; Shigella; Francisella tularensis; Xanthomonas campestris (plants) or Salmonella), thus, causing an infection. In some embodiments, the bacteria may grow, multiply, and/or produce toxins in the individual's cells, tissues, and/or organs. In some embodiments, a bacterial infection can be any situation in which the presence of a bacteria population(s) is latent within or damaging to a host body. Thus, an individual is “suffering” from a bacterial infection when a latent bacterial population is detectable in or on the individual's body, an excessive amount of a bacterial population is present in or on the individual's body, or when the presence of a bacterial population(s) is damaging to the cells, tissues, and/or organs of the individual.

As used herein, the terms “protecting against a bacterial infection” and “preventing a bacterial infection” are used interchangeably and refer to preventing an individual from developing a bacterial infection or decreasing the risk that an individual may develop a bacterial infection (e.g., Brucella; Campylobacter spp.; Bartonella henselae; Vibrio cholerae; Escherichia coli; Haemophilus influenzae; Klebsiella; Enterobacter; Serratia; Legionella pneumophila; Bordetella pertussis; Yersinia pestis; Pseudomonas spp.; Salmonella; Shigella; Francisella tularensis; or Salmonella). Prophylactic drugs used in methods of protecting against a bacterial infection in an individual are often administered to the individual prior to any detection of the bacterial infection. In some embodiments of methods of protecting against a bacterial infection, an individual (e.g., an individual at risk of developing a bacterial infection) may be administered a pharmaceutical composition of the invention to prevent the bacterial infection development or decrease the risk of the bacterial infection development.

As used herein, the terms “treating” and “to treat” are used interchangeably and refer to a therapeutic treatment of a bacterial infection in an individual. In some embodiments, a therapeutic treatment may slow the progression of the bacterial infection, improve the individual's outcome, and/or eliminate the infection. In some embodiments, a therapeutic treatment of a bacterial infection in an individual may alleviate or ameliorate of one or more symptoms or conditions associated with the bacterial infection, diminish the extent of the bacterial infection, stabilize (i.e., not worsening) the state of the bacterial infection, prevent the spread of the bacterial infection, and/or delay or slow the progress of the bacterial infection, as compared to the state and/or the condition of the bacterial infection in the absence of therapeutic treatment.

As used herein, the term “effective amount” is meant the amount of drug (e.g., a composition comprising a tunicamycin derivative or salt thereof and a polymyxin compound or salt thereof) required to treat or prevent a bacterial infection or a disease associated with a bacterial infection in an individual. The effective amount of drug used to practice the methods described herein for therapeutic or prophylactic treatment of conditions caused by or contributed to by a bacterial infection varies depending upon the manner of administration, the age, body weight, and general health of the individual. Ultimately, the attending practitioner will decide the appropriate amount and dosage regimen. Such amount is referred to as an “effective amount.”

As used herein, the term “salt” refers to any pharmaceutically-acceptable salt, such as a non-toxic acid addition salt, metal salt, or metal complex, commonly used in the pharmaceutical industry. Examples of acid addition salts include organic acids, such as acetic, lactic, palmoic, maleic, citric, cholic acid, capric acid, caprylic acid, lauric acid, glutaric, glucuronic, glyceric, glycocolic, glyoxylic, isocitric, isovaleric, lactic, malic, oxaloacetic, oxalosuccinic, propionic, pyruvic, ascorbic, succinic, benzoic, palmitic, suberic, salicylic, tartaric, methanesulfonic, toluenesulfonic, and trifluoroacetic acids, and inorganic acids, such as hydrochloric acid, hydrobromic acid, sulfuric acid, and phosphoric acid. Representative alkali or alkaline earth metal salts include sodium, lithium, potassium, calcium, and magnesium, among others.

As used herein, the term “pharmaceutically acceptable” refers to those compounds, materials, compositions, and/or dosage forms, which are suitable for contact with the tissues of an individual (e.g., an animal or a human), without excessive toxicity, irritation, allergic response and other problem complications commensurate with a reasonable benefit/risk ratio.

As used herein, the term “pharmaceutical composition” refers to a mixture containing a therapeutic compound to be administered to a subject (e.g., an animal, a human, or a plant), in order to prevent, treat, or control a particular disease or condition affecting the subject, such as a bacterial infection.

As used herein, the term “excipient” refers to a substance formulated alongside the active ingredient of a pharmaceutical composition. At least one excipient may be included, for example, for the purpose of long-term stabilization, or to confer a therapeutic enhancement on the active ingredient in the final dosage form.

As used herein, the term “between” refers to any quantity within the range indicated and enclosing each of the ends of the range indicated.

Other than in the operating examples, or where otherwise indicated, all numbers expressing quantities of ingredients, concentrations, or reaction conditions used herein should be understood as modified in all instances by the term “about.”

As used herein, the term “about” is defined as plus or minus ten percent of a recited value. For example, about 1.0 g means 0.9 g to 1.1 g.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicate otherwise.

Mention of trade names or commercial products in this disclosure is solely for the purpose of providing specific information and does not imply recommendation or endorsement by the U.S. Department of Agriculture.

Embodiments of the present invention are shown and described herein. It will be obvious to those skilled in the art that such embodiments are provided by way of example only. Numerous variations, changes, and substitutions will occur to those skilled in the art without departing from the invention. Various alternatives to the embodiments of the invention described herein may be employed in practicing the invention. It is intended that the included claims define the scope of the invention and that methods and structures within the scope of these claims and their equivalents are covered thereby. All publications, patents, and patent applications mentioned in this specification are herein incorporated by reference to the same extent as if each individual publication, patent, or patent application was specifically and individually indicated to be incorporated by reference.

### EXAMPLES

Having now generally described this invention, the same will be better understood by reference to certain specific examples, which are included herein only to further illustrate the invention and are not intended to limit the scope of the invention as defined by the claims.

### Example 1 Polymyxin Antimicrobial Activity

The antimicrobial activity of polymyxin in the presence or absence of tunicamycin or a tunicamycin derivative was tested against Bacillus subtilis (Gram-positive bacterium), Saccharomyces cerevisiae (eukaryote), Escherichia coli (Gram-negative bacterium), and Pseudomonas aeruginosa (Gram-negative bacterium) in an in vitro assay.

The mycobacterial growth inhibition (MIC) using natural tunicamycin (TUN), a tunicamycin derivative TunR2, polymyxin B, or a combination of polymyxin B with TUN or TunR2 were tested. Testing was performed using the resazurin staining assay. The resazurin staining assay uses the reduction by bacterial viable cells of the blue, non-fluorescent, and non-toxic dye, resazurin, to pink, fluorescent resorufin to assess the viability of biofilms. The Minimal Inhibitory Concentration (MIC) values where determined using standard microbiological methods, for example by spotting dilutions of the compounds or combinations on solid agar media plates after inoculating with bacteria, yeast, or buffer alone, followed by incubating for a defined period. This method results in clearing zones on agar plates.

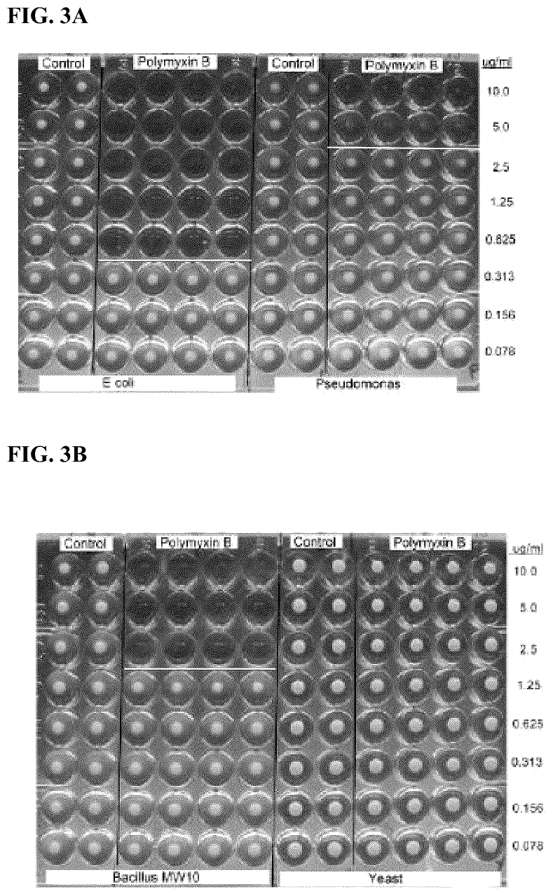

To determine the MIC for polymyxin B alone, plates inoculated with E. coli MG1655 (Gram-negative bacterium), P. aeruginosa ATCC 2783 (Gram-negative bacterium), B. subtilis MW10 (Gram-positive bacterium), S. cerevisiae (eukaryote) in TYG, or DMSO alone (Control) were spotted with a dilution gradient of polymyxin B, and incubated for 24 hours at 30° C. As seen in FIG. 3A and FIG. 3B, the MIC for polymyxin B alone was 0.625 μg/mL for E. coli, 5.0 μg/mL for P. aeruginosa, 2.5 μg/mL for B. subtilis, and the effect on yeast was not detectable.

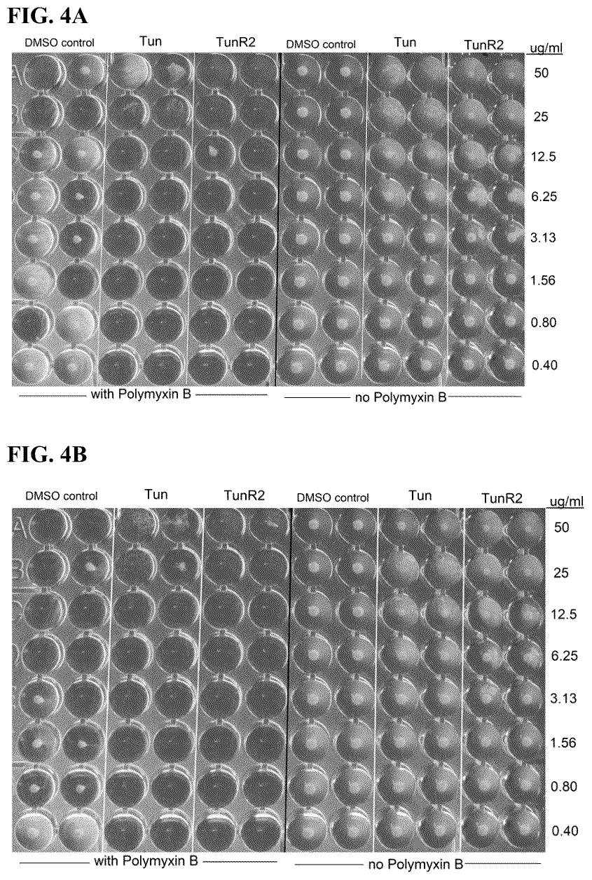

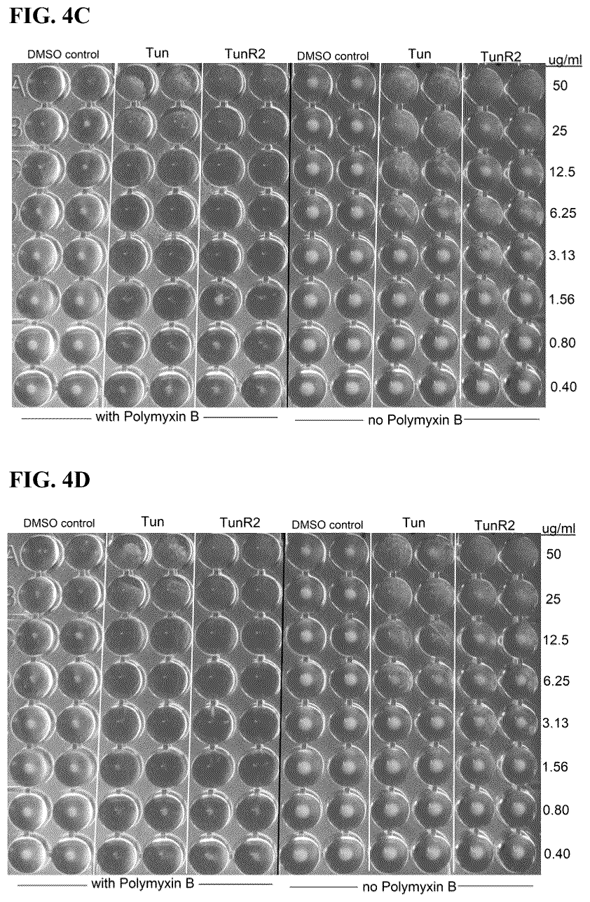

To determine the MIC for TUN or TunR2 in the presence or absence of polymyxin B, plates inoculated with E. coli MG1655, or P. aeruginosa ATCC 2783 in TYG, were spotted with a dilution gradient of Tun or TunR2 in the presence or absence of polymyxin B at sub-MIC concentration (MIC/2), or DMSO alone (Control). Images of plates after incubation for 24 hours at 37° C. are shown in FIG. 4A to FIG. 4D. Images of two plates inoculated with E. coli are shown in FIG. 4A and FIG. 4B, and images of two plates inoculated with P. aeruginosa are shown in FIG. 4C and FIG. 4D. As seen in the four right-hand columns of wells in FIG. 4A to FIG. 4D, after incubation for 24 hours at 37° C., in the absence of polymyxin B neither Tun nor TunR2 had noticeable effect on E. coli or P. aeruginosa. But, as seen in columns three to six of FIG. 4A to FIG. 4D, no E. coli or P. aeruginosa grew after incubation for 24 hours at 37° C. when in the presence of polymyxin B and Tun or polymyxin B and TunR2.

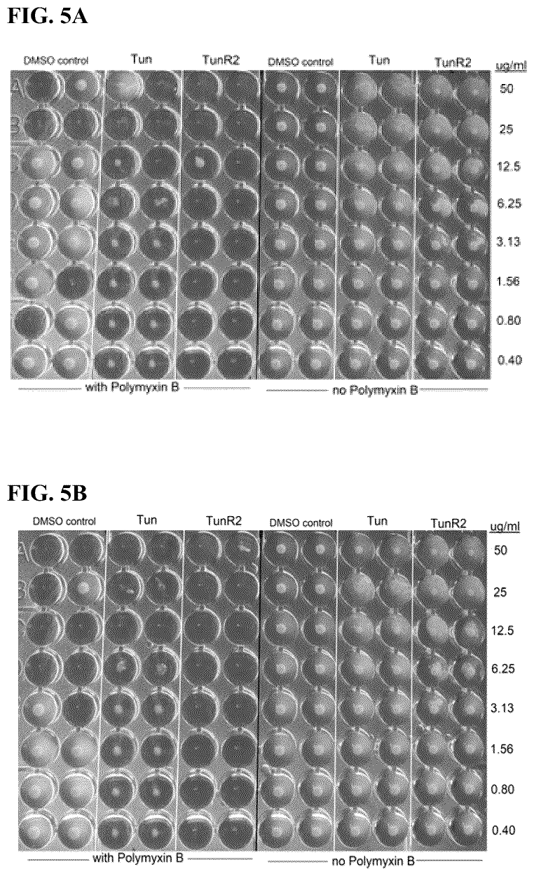

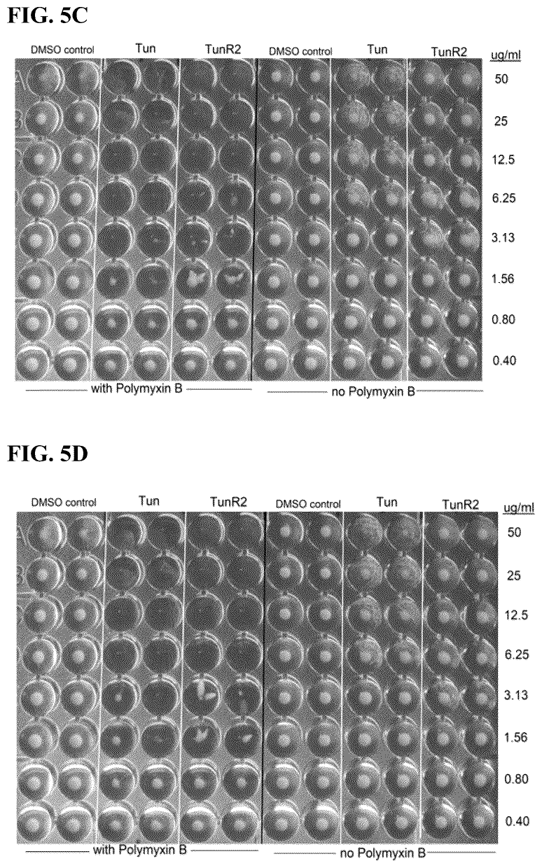

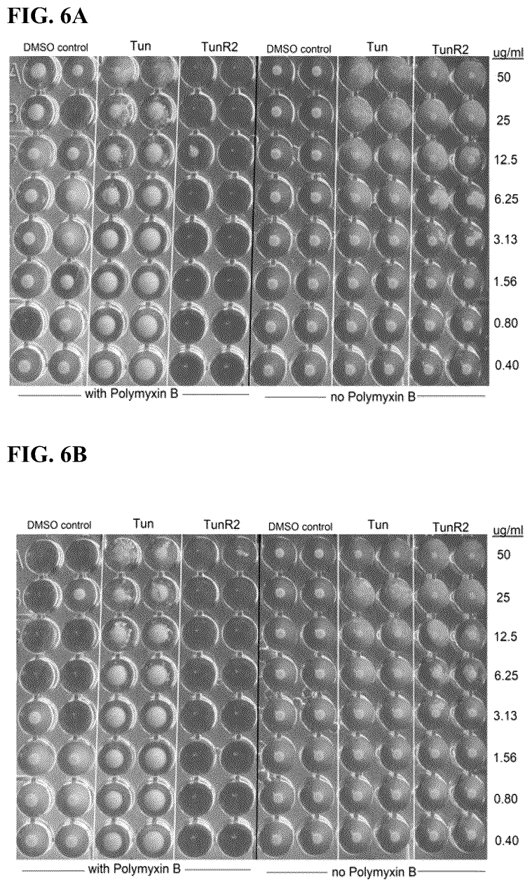

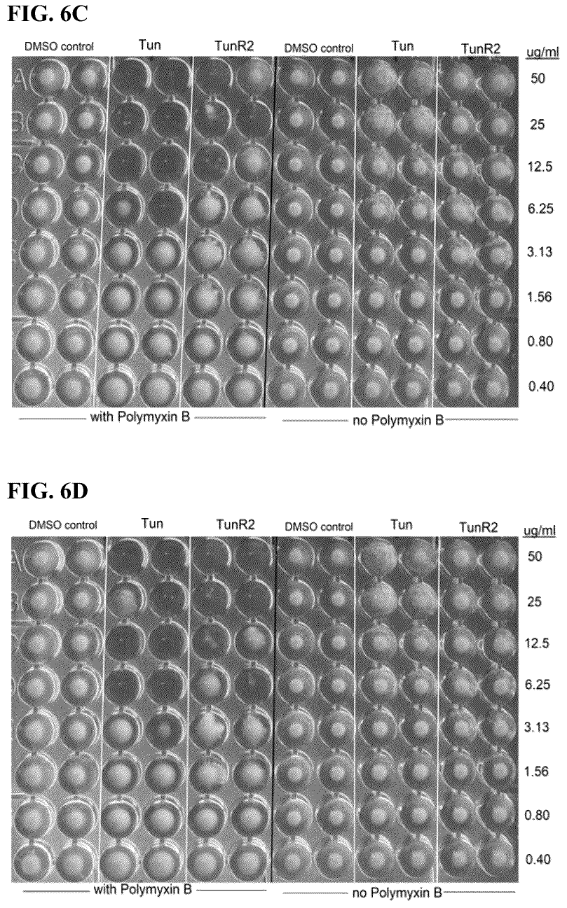

Images of plates after incubation for 48 hours at 37° C. are shown in FIG. 5A to FIG. 5D. Images of two plates inoculated with E. coli are shown in FIG. 5A and FIG. 5B, and images of two plates inoculated with P. aeruginosa are shown in FIG. 5C and FIG. 5D. As seen in these images, in the presence of sub-MIC polymyxin B concentration, the MIC for TunR2 for E. coli was less than 0.4 μg/mL, and was 3.13 μg/mL for P. aeruginosa. Images of plates after incubation for 7 days at 37° C. are shown in FIG. 6A to FIG. 6D. Images of two plates inoculated with E. coli are shown in FIG. 6A and FIG. 6B, and images of two plates inoculated with P. aeruginosa are shown in FIG. 6C and FIG. 6D. As seen in these images, in the presence of sub-MIC polymyxin B concentration, the MIC for TunR2 for E. coli was less than 0.4 μg/mL, and was 6.12 μg/mL for P. aeruginosa.

This Example shows a surprising synergistic effect when using TUN or TunR2 in the presence of a sub-MIC concentration of polymyxin B on Gram-negative bacteria.

### Example 2 Activity Against Other Gram-Negative Pathogens

The polymyxin B synergistic effect on natural tunicamycin (TUN) or TunR2 on Gram-negative bacteria were tested for other pathogens.

The activity of TUN or TunR2 in the presence or absence of polymyxin B on Serratia marcescens (a honey bee pathogen) or E. coli was tested. The MIC was determined using the resazurin staining assay described in Example 1.

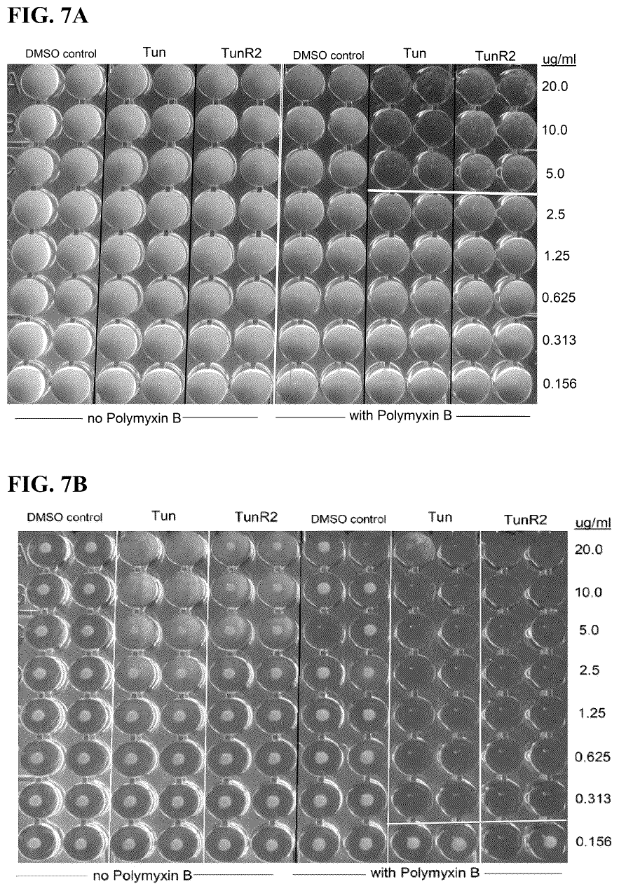

Plates inoculated with S. marcescens B-2544, E. coli, or DMSO alone (Control) in TYG were spotted with a dilution gradient of TUN or TunR2 in the presence or absence of a sub-MIC concentration of polymyxin B (half the MIC concentration), and incubated for 24 hours at 37° C. An image of a plate inoculated with S. marcescens is shown in FIG. 7A, and an image of a plate inoculated with E. coli is shown in FIG. 7B. In each of the plates well columns 1 and 2 were treated with DMSO alone, columns 3 and 4 were treated with Tun in the absence of polymyxin B, columns 5 and 6 were treated with TunR2 in the absence of polymyxin B, columns 7 and 8 were treated with DMSO in the presence of polymyxin B, columns 9 and 10 were treated with TUN in the presence of polymyxin B, and columns 11 and 12 were treated with TunR2 in the presence of polymyxin B. As seen in FIG. 7A, when used alone neither TUN nor TunR2 had a detectable effect on S. marcescens at any of the concentrations tested, but they were both effective when in the presence of polymyxin B with a MIC of 5.0 μg/mL. As seen in FIG. 7B, when used alone neither TUN nor TunR2 had a detectable effect on E. coli at any of the concentrations tested, but they were both effective when in the presence of polymyxin B with a MIC of 0.313 μg/mL. The results obtained for E. coli compare well with the MICs of other antibiotics as taught by m. de Boer et al. (2015, “Minimum Inhibitory Concentrations of Selected Antimicrobials Against Escherichia Coli and Trueperella pyogenes of Bovine Uterine Origin,” J Dairy Sci. 98:4427-4438).

This Example shows that the surprising synergistic effect when using TUN or TunR2 in the presence of a sub-MIC concentration of polymyxin B may be extended to other Gram-negative bacteria.

### Example 3 Three-Way Combinations

The effect of TunR2/PmB combinations on a β-lactam antibiotic was tested on plates inoculated with P. aeruginosa.

The activity of Cefquinome alone, or in the presence of TunR2, polymyxin B, or polymyxin B and TunR2 on P. aeruginosa was tested. The MIC was determined using the resazurin staining assay described in Example 1.

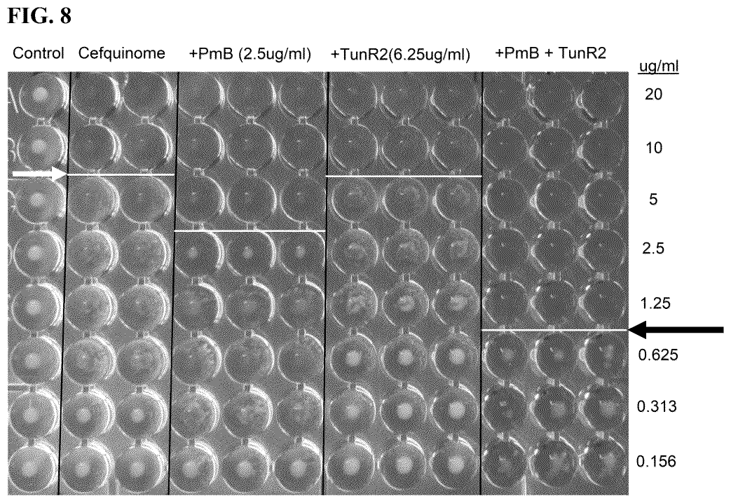

Plates inoculated with P. aeruginosa, or DMSO alone (Control) in TYG were spotted with a dilution gradient of Cefquinome in the presence of TunR2, polymyxin B, or polymyxin B and TunR2, and incubated for 24 hours at 37° C. As seen in FIG. 8, the MIC for Cefquinome alone was 10 μg/mL; the MIC for Cefquinome in the presence of 2.5 μg/mL polymyxin B was 5 μg/mL; the MIC for Cefquinome in the presence of 6.25 μg/mL TunR2 was 10 μg/mL; and the MIC in the presence of 2.5 μg/mL polymyxin B and 6.25 μg/mL TunR2 was enhanced to 1.25 μg/mL.

This Example shows a surprising synergistic effect when using Cefquinome in the presence of TunR2 and a sub-MIC concentration of polymyxin B.

We claim:

1. An antibacterial composition comprising at least one polymyxin and at least one tunicamycin or salt thereof, or comprising at least one polymyxin and at least one modified tunicamycin or salt thereof.

2. The antibacterial composition of claim 1, wherein the modified tunicamycin or salt thereof comprises Formula I 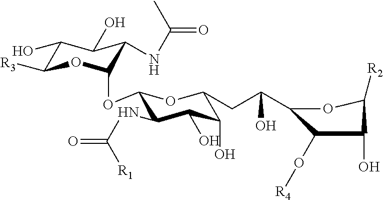
wherein R₁ is independently
(i) CH₃-(CH₂)ₙ-CH₂-CH₂- and n is independently any integer from 9 to 15, or
(ii) CH₃-CH₂-CH(CH₃)(CH₂)ₙ-CH₂-CH₂- and n is independently any integer from 6 to 12;
wherein R₂ is X or Y 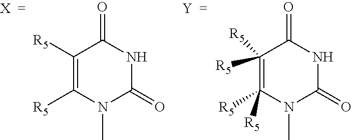
wherein R₃ is independently HOCH₂-, an alkyl hydrocarbon, or a non-alkyl C₁₋₁₀ hydrocarbon;
wherein R₄ is independently H or -P(O)(OH)₂; and
wherein R₅ is independently H, F, Cl, Br, I, an alkyl hydrocarbon, or a non-alkyl C₁₋₁₀ hydrocarbon; or
wherein the modified tunicamycin or salt thereof is a single-or double-reduced tunicamycin of Formula II 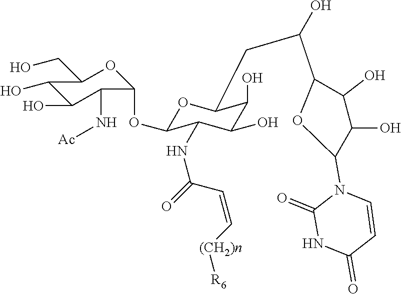
wherein the fatty acid acyl 2″′, 3″′-double bond and/or the uracil 5,6 double bond are reduced,
n is independently any integer from 7 to 13; and
R₆ is a C1 or C2 straight chain, an iso-branched chain, an anteiso-branched chain, a tert-butyl-branched chain, a cyclopropane, a cyclobutene, a cyclopentane, a cyclohexane, a cycloheptane, an unsaturated cyclohexene, or an unsaturated cyclopentene.

3. The antibacterial composition of claim 1, wherein the at least one polymyxin is polymyxin B or polymyxin E.

4. The antibacterial composition of claim 1, further comprising a β-lactam antibiotic, a non-β-lactam antibiotic, or a combination thereof.

5. The antibacterial composition of claim 4, wherein the antibacterial composition comprises a β-lactam antibiotic, and the β-lactam antibiotic is a penicillin, a cephalosporin, a monobactam, a carbapenem, or a combination thereof.

6. A method of inhibiting or treating a bacterial infection caused by Gram-negative bacteria in a subject in need thereof, the method comprising administering to the subject a therapeutically effective amount of the antibacterial composition of claim 1.

7. The method of claim 6, wherein the modified tunicamycin or salt thereof comprises Formula I 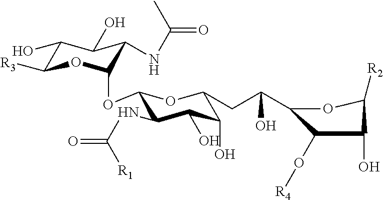
wherein R₁ is independently
(i) CH₃-(CH₂)ₙ-CH₂-CH₂- and n is independently any integer from 9 to 15, or
(ii) CH₃-CH₂-CH(CH₃)(CH₂)ₙ-CH₂-CH₂- and n is independently any integer from 6 to 12;
wherein R₂ is X or Y 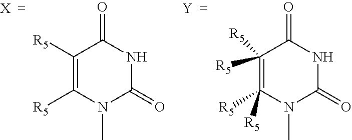
wherein R₃ is independently HOCH₂-, an alkyl hydrocarbon, or a non-alkyl C₁₋₁₀ hydrocarbon; wherein R₄ is independently H or P-(O)(OH)₂; and
wherein R₅ is independently H, F, Cl, Br, I, an alkyl hydrocarbon, or a non-alkyl C₁₋₁₀ hydrocarbon; or
wherein the modified tunicamycin or salt thereof comprises a single- or double-reduced tunicamycin of Formula II 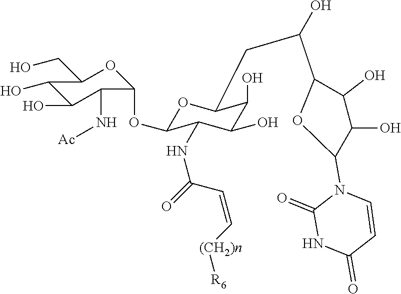
wherein the fatty acid acyl 2″′, 3″′-double bond and/or the uracil 5,6 double bond are reduced,
n is independently any integer from 7 to 13; and
R₆ is a C1 or C2 straight chain, an iso-branched chain, an anteiso-branched chain, a tert-butyl-branched chain, a cyclopropane, a cyclobutene, a cyclopentane, a cyclohexane, a cycloheptane, an unsaturated cyclohexene, or an unsaturated cyclopentene.

8. The method of claim 6, wherein the at least one polymyxin is polymyxin B or polymyxin E.

9. The method of claim 6, further comprising β-lactam antibiotic, a non-β-lactam antibiotic, or a combination thereof.

10. The method of claim 9, wherein the antibacterial composition comprises a β-lactam antibiotic, and the β-lactam antibiotic is a penicillin, a cephalosporin, a monobactam, a carbapenem, or a combination thereof.

11. A method of killing Gram-negative bacteria in or on a subject, the method comprising administering to the subject an effective amount of the antibacterial composition of claim 1.

12. The method of claim 11, wherein the modified tunicamycin or salt thereof comprises Formula I 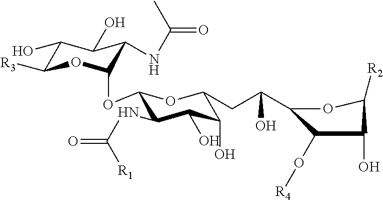
wherein R₁ is independently
(i) CH₃-(CH₂)ₙ-CH₂-CH₂- and n is independently any integer from 9 to 15, or
(ii) CH₃-CH₂-CH(CH₃)(CH₂)ₙ-CH₂-CH₂- and n is independently any integer from 6 to 12;
wherein R₂ is X or Y 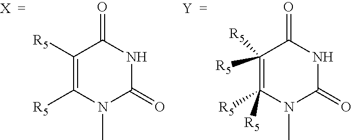
wherein R₃ is independently HOCH₂-, an alkyl hydrocarbon, or a non-alkyl C₁₋₁₀ hydrocarbon; wherein R₄ is independently H or P-(O)(OH)₂; and
wherein R₅ is independently H, F, Cl, Br, I, an alkyl hydrocarbon, or a non-alkyl C₁₋₁₀ hydrocarbon; or
wherein the modified tunicamycin or salt thereof comprises a single- or double-reduced tunicamycin of Formula II 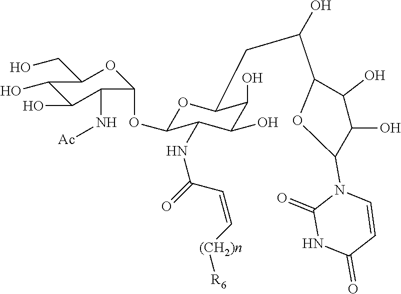
wherein the fatty acid acyl 2″′, 3″′-double bond and/or the uracil 5,6 double bond are reduced,
n is independently any integer from 7 to 13; and
R₆ is a C1 or C2 straight chain, an iso-branched chain, an anteiso-branched chain, a tert-butyl-branched chain, a cyclopropane, a cyclobutene, a cyclopentane, a cyclohexane, a cycloheptane, an unsaturated cyclohexene, or an unsaturated cyclopentene.

13. The method of claim 11, wherein the at least one polymyxin is polymyxin B or polymyxin E.

14. The method of claim 11, further comprising a β-lactam antibiotic, a non-β-lactam antibiotic, or a combination thereof.

15. The method of claim 14, wherein the antibacterial composition comprises a β-lactam antibiotic, and the β-lactam antibiotic is a penicillin, a cephalosporin, a monobactam, a carbapenem, or a combination thereof.

16. A method of disinfecting an object or a surface that has Gram-negative bacteria on the object or the surface, the method comprising applying an effective amount of the antibacterial composition of claim 1 to the object or the surface to kill the Gram-negative bacteria present on the object or the surface.

17. The method of claim 16, wherein the at least one polymyxin is polymyxin B or polymyxin E.

18. The method of claim 16, further comprising a β-lactam antibiotic, a non-β-lactam antibiotic, or a combination thereof.

19. The method of claim 18, wherein the antibacterial composition comprises β-lactam antibiotic, and the β-lactam antibiotic is a penicillin, a cephalosporin, a monobactam, a carbapenem, or a combination thereof.
